(19) United States
(12) Patent Application Publication
(10) Pub. No.: US 2007/0041952 A1
(43) Pub. Date: Feb. 22, 2007

(54) Three-dimensional fiber scaffolds for tissue engineering

(75) Inventors: Farshid Guilak, Durham, NC (US); Franklin T. Moutos, Raleigh, NC (US)

Correspondence Address: JENKINS, WILSON, TAYLOR & HUNT, P. A., 3100 TOWER BLVD, SUITE 1200, DURHAM, NC, 27707, US

(73) Assignee: Duke University, Durham, NC (US)

(21) Appl. No.: 11/406,519

(22) Filed: Apr. 18, 2006

Related U.S. Application Data
(60) Provisional application No. 60/672,344, filed on Apr. 18, 2005.
(60) Provisional application No. 60/780,952, filed on Mar. 10, 2006.

(51) Int. Cl. A61K 35/12; A61F 2/06

(52) U.S. Cl. 424/93.7; 424/422; 623/1.11

(57) ABSTRACT

A tissue restoration implant adapted for use with a pre-determined tissue, and methods of making and using the same. The tissue restoration implant adapted for use with a pre-determined tissue can include a three-dimensional fiber scaffold, the scaffold comprising at least three systems of fibers; wherein two of the three fiber systems define an upper layer, a lower layer and a medial layer between the upper layer and the lower layer within the three-dimensional fiber scaffold; wherein one of the at least three fiber systems interconnects the upper layer, the lower layer and the medial layer; wherein the at least three fiber systems each comprise a bio-compatible material; and wherein the fiber scaffold, or one or more of the fiber systems, provide a characteristic that functions to restore the pre-determined tissue upon implantation. The tissue restoration implant adapted for use with a pre-determined tissue can include one or more cells that can develop into the pre-determined tissue.

### RELATED APPLICATIONS

This application claims the benefit of U.S. Provisional Patent Application Ser. No.60/672,344, filed Apr. 18, 2005 and further claims the benefit of U.S. Provisional Patent Application Ser. No. 60/780,952 filed Mar. 10, 2006, the disclosures of which are incorporated herein by reference in their entireties.

### GOVERNMENT INTEREST

This invention was made with U.S. Government support under NIH Grant Nos. AR49294. Accordingly, the U.S. Government has certain rights in the present subject matter.

### TECHNICAL FIELD

The presently disclosed subject matter relates to a three-dimensional fiber scaffold for tissue engineering. The scaffold can provide a characteristic that functions to restore a tissue upon implantation, and representative characteristics include, but are not limited to, inhomogeneity, anisotropy, non-linearity, viscoelasticity, and combinations thereof.

### ABBREVIATIONS

<—less than
>—greater than
±—plus or minus
%—percent
°—degrees
μm—micrometer
ε_{z}—axial strain
v—Poisson's ratio
γ—shear strain
ω—angular frequency
σ—tensile stress
2-D—two-dimensional
3-D—three-dimensional
A—area
ANOVA—analysis of variance
E—strain
E₀—0% strain
ECM—extracellular matrix
F—force
h—thickness
H_{A}—compressive modulus
k—hydraulic permeability
KPa—kilopascal
m—meter
mg—milligram
ml—milliliter
MPa—millipascal
N—Newton
p—probability
PGA—polyglycolic acid
rad—radian
s—second
SEM—standard error of the mean
vs.—versus

### BACKGROUND

Tissue engineering is a relatively new but rapidly growing discipline wherein living cells are used to replace functional tissue loss due to injury, disease, or birth defect in an animal or human. The field of tissue engineering has sought to use combinations of implanted cells, biomaterials, and biologically active molecules to restore, repair, and/or regenerate injured or diseased tissues. Despite many advances, significant challenges remain in restoring tissues, including particularly those tissues that serve a predominantly biomechanical function, such as articular cartilage.

Articular cartilage is the smooth, wear-resistant surface that lines the ends of bones in diarthrodial joints and serves to support and distribute applied loads (Guilak, F., Setton, L. A., and Kraus, V. B. (2000) In Principles of Practice of Orthopaedic Sports Medicine (ed. K. P. Speer W. E. Garrett Jr., and D. T. Kirkendall) pp. 53-73 (Lippincott Williams and Wilkins, Philadelphia; Mow, V. C., Ratcliffe, A., & Poole, A. R. (1992) Biomaterials 13:67-97). Accordingly, the function of articular cartilage is to provide a low friction surface enabling the joint to withstand weight bearing through the range of motion needed to perform activities of daily living and athletic endeavors, such as walking, stair climbing, and work-related activities.

Presently, articular cartilage repair remains an important and unsolved clinical problem, and a number of recent studies have applied tissue engineering approaches in an effort to promote cartilage regeneration. Despite numerous advances, challenges still remain in the development of a tissue-engineered replacement that restores the complex biomechanical properties of articular cartilage. From a biomechanical standpoint, this tissue can be represented as a multiphasic fiber-reinforced material, with anisotropic, inhomogeneous, nonlinear, and viscoelastic properties (Mow, V. C., et al., (1980) J. Biomech. Engng. 102:73; Soltz, M. A., Ateshian, G. A. (2000) J. Biomech. Engng. 122:576; Woo, S. L., et al. (1979) J. Biomech. 12:437).

Most previous tissue engineering approaches have utilized scaffolds comprised of highly porous meshes or hydrogels that are relatively isotropic and thus cannot provide the complex multidirectional and nonlinear properties believed necessary for sustained load support in vivo (Soltz, M. A., Ateshian, G. A. (2000) J. Biomech. Engng. 122:576). Traditional textile reinforced composites are made with 2-dimensional (2-D) woven fabrics. Ordinary 2-D weaving processes mechanically interlock yarns perpendicularly to each other by bending or crimping, significantly reducing each fiber's strength and subsequently, the reinforcement properties of the fabric. Additionally, composite parts, which require substantial thickness or complex shapes, must be made from multiple layers of fabric and/or fabrics cut and sewn to create the desired geometry. These labor-intensive processes introduce variability and broken fiber ends into the finished composites and result in substantial reduction in composite performance.

Notably, the mechanical properties of prior art scaffolds, particularly stiffness and strength, are several orders of magnitude lower than those of native cartilage (Pei, M., et al. (2002) Faseb J 16:1691-1694; Mauck, R. L. et al. (2000) J Biomech Eng 122:252-260; LeRoux, M. A., Guilak, F. and Setton, L. A. (1999) J Biomed Mater Res 47:46-53; Smidsrod, O. and Skjak-Braek, G. (1990) Trends Biotechnol8:71-78), thus requiring prolonged in vitro culture to attain native tissue properties before implantation.

Therefore, there is a need in the art to identify structural and mechanical properties of replacement tissues that are critical in restoring functionality to the repaired site, and to incorporate these criteria into the design and manufacture of new engineered tissue constructs. The presently disclosed subject matter addresses this and other needs in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter describes a tissue restoration implant comprising a three-dimensional fiber scaffold that can be used in tissue repair, restoration, and/or regeneration. The presently disclosed subject matter further methods of producing the tissue restoration implant comprising providing a three-dimensional fiber scaffold and implanting at a pre-determined site so as to restore the pre-determined tissue upon implantation of the tissue restoration implant. The three-dimensional scaffold can provide a characteristic that functions to restore a tissue upon implantation, including, but not limited to, inhomogeneity, anisotropy, non-linearity, viscoelasticity, and combinations thereof.

In some embodiments, the presently disclosed subject matter provides a tissue restoration implant adapted for use with a pre-determined tissue. The tissue restoration implant comprises a three-dimensional fiber scaffold, the scaffold comprising at least three systems of fibers; wherein two of the three fiber systems define an upper layer, a lower layer and a medial layer between the upper layer and the lower layer within the three-dimensional fiber scaffold; wherein one of the at least three fiber systems interconnects the upper layer, the lower layer and the medial layer; wherein the at least three fiber systems each comprise a bio-compatible material; and wherein the fiber scaffold, or one or more of the fiber systems, provide a characteristic that functions to restore the pre-determined tissue upon implantation.

In some embodiments, the three-dimensional fiber scaffold further comprises one or more cells that can develop into the pre-determined tissue.

In some embodiments, a method of producing a tissue restoration implant for use in tissue restoration is provided. The method comprises forming a three-dimensional fiber scaffold with at least three fiber systems such that two of the three fiber systems define an upper layer, a lower layer, and a medial layer between the upper layer and the lower layer within the three-dimensional fiber scaffold, wherein one of the at least three fiber systems interconnects the upper layer, the lower layer, and the medial layer, wherein the at least three fiber systems each comprise a biocompatible material, and wherein the fiber scaffold, or one or more of the fiber systems, provide a characteristic that functions to restore the pre-determined tissue upon implantation, whereby a tissue restoration implant is produced.

In some embodiments, a method of producing a tissue restoration implant for use in tissue restoration is provided wherein the method comprises (a) providing a three-dimensional fiber scaffold formed of at least three systems of fibers, wherein two of the three fiber systems define an upper layer, a lower layer, and a medial layer between the upper layer and the lower layer within the three-dimensional fiber scaffold, wherein one of the at least three fiber systems interconnects the upper layer, the lower layer, and the medial layer, wherein the at least three fiber systems each comprise a biocompatible material, and wherein the fiber scaffold, or one or more of the fiber systems, provide a characteristic that functions to restore the pre-determined tissue upon implantation; and (b) implanting at a pre-determined site in the subject the three-dimensional fiber scaffold provided in (a) to thereby restore a tissue in the subject.

In some embodiments, the three-dimensional fiber scaffold comprises three orthogonally woven fiber systems, a plurality of braided fiber systems, a plurality of circular woven fiber systems, or combinations thereof.

In some embodiments, one of the three orthogonally woven fiber systems is inserted into the scaffold as a single fiber and severed at a pre-determined point.

In some embodiments, the biocompatible material comprises a material selected from the group consisting of an absorbable material, a non-absorbable material, and combinations thereof.

In some embodiments, the non-absorbable material is selected from the group including, but not limited to, polypropylene, polyester, polytetrafluoroethylene (PTFE), expanded PTFE (ePTFE), polyethylene, polyurethane, polyamide, nylon, polyetheretherketone (PEEK), polysulfone, a cellulosic, fiberglass, an acrylic, tantalum, polyvinyl alcohol, carbon, ceramic, a metal, and combinations thereof.

In some embodiments, the absorbable material is selected from the group including, but not limited to, polyglycolic acid (PGA), polylactic acid (PLA), polyglycolide-lactide, polycaprolactone, polydioxanone, polyoxalate, a polyanhydride, a poly(phosphoester), catgut suture, collagen, silk, chitin, chitosan, hydroxyapatite, bioabsorbable calcium phosphate, hyaluronic acid, elastin, and combinations thereof.

In some embodiments, the fiber systems further comprise a monofilament fiber, a multifilament fiber, a hollow fiber, a fiber having a variable cross-section along its length, or a combination thereof.

In some embodiments, the at least three fiber systems in at least one of the upper, medial and lower layers define a plurality of interstices within the fiber scaffold.

In some embodiments, the interstices further comprise a pore size ranging from about 10 μm to about 250 μm.

In some embodiments, the interstices further comprise a pore size ranging from about 25 μm to about 175 μm.

In some embodiments, the interstices further comprise a pore size ranging from about 50 μm to about 125 μm.

In some embodiments, the characteristic that functions to restore the pre-determined tissue upon implantation is selected from the group consisting of inhomogeneity, anisotropy, nonlinearity, viscoelasticity, and combinations thereof.

In some embodiments, the one or more cells that can develop into a pre-determined tissue are present in a matrix. In some embodiments, the matrix comprises a gel.

In some embodiments, the one or more cells are selected from the group consisting of primary cells, undifferentiated progenitor cells, chondrocytes, bone-precursor cells, stem cells, cells of the periosteum, or perichondrium tissue, and combinations thereof.

In some embodiments, the pre-determined tissue is articular cartilage.

In some embodiments, the tissue restoration implant comprises a cell growth modulating material.

In some embodiments, the cell growth modulating material is selected from the group consisting of a growth factor, a cytokine, a chemokine, a collagen, gelatin, laminin, fibronectin, thrombin, lipids, cartilage oligomeric protein (COMP), thrombospondin, fibrin, fibrinogen, Matrix-GLA (glycine-leucine-alanine) protein, chondrocalcin, tenascin, a mineral, an RGD (arginine, glycine, aspartic acid) peptide or RGD-peptide containing molecule, elastin, hyaluronic acid, a glycosaminoglycans, a proteoglycan, water, an electrolyte solution, and combinations thereof.

Accordingly, it is an object of the presently disclosed subject matter to provide a tissue restoration implant comprising a three-dimensional fiber scaffold for use in tissue engineering and methods of making and using such implants. This object is achieved in whole or in part by the presently disclosed subject matter.

An object of the presently disclosed subject matter having been stated hereinabove, other objects and advantages will become apparent to those of ordinary skill in the art after a study of the following description of the presently disclosed subject matter and non-limiting examples.

### BRIEF DESCRIPTION OF THE DRAWINGS

FIG. 1A is a schematic representation of a weaving loom for use in accordance with the presently disclosed subject matter.

FIG. 1B is a perspective view of a weaving loom for use in accordance with the presently disclosed subject matter.

FIG. 2A is a schematic representation of the unit cell of a three-dimensional orthogonally woven structure.

FIG. 2B is a surface scanning electron micrograph (SEM) (40×) view of the three-dimensional orthogonally woven structure in the X-Y plane.

FIG. 2C is a cross-sectional SEM (40×) view of the three-dimensional orthogonally woven structure in the Y-Z plane.

FIG. 2D is a cross-sectional SEM (40×) view of the three-dimensional orthogonally woven structure in the X-Z plane.

FIG. 3 is a fluorescent calcein-AM labeled digital image of a construct freshly seeded with porcine articular chondrocytes in 2% agarose, showing a spatially uniform initial distribution of cells with rounded morphology within the 3-D orthogonally woven structure.

FIGS. 4A-4D are a series of bar graphs that represent aggregate modulus (H_{A}), Young's modulus (E), hydraulic permeability (k), shear modulus (G*), and equilibrium shear modulus (G), as determined by confined and unconfined compression of the 3D orthogonally woven structure, respectively.

FIG. 4A is a set of bar graphs indicating that fiber-reinforced composite scaffolds (solid bars) show significantly higher aggregate and Young's moduli than scaffolds with unreinforced agarose (diagonal bars).

FIG. 4B is a set of bar graphs indicating that scaffolds woven with small pores show significantly higher aggregate moduli than large pore scaffolds under confined compression. Scaffolds woven with 2% agarose-small pores are represented by grey bars, scaffolds woven with 2% agarose-large pores are represented by left-diagonal bars, scaffolds woven with 3% agarose-small pores are represented by solid black bars, scaffolds woven with 3% agarose-large pores are represented by right-diagonal bars, scaffolds woven with fibrin-small pores are represented by open bars, and scaffolds woven with fibrin-large pores are represented by cross-hatched bars. Data presented are mean ±SEM, and *p<0.005.

FIG. 4C is a bar graph illustrating hydraulic permeability (k) of composite scaffolds determined by curve-fitting creep tests using a nonlinear numerical least squares regression procedure. Scaffolds woven with 2% agarose-small pores are represented by grey bars, scaffolds woven with 2% agarose-large pores are represented by left-diagonal bars, scaffolds woven with 3% agarose-small pores are represented by solid black bars, scaffolds woven with 3% agarose-large pores are represented by right-diagonal bars, scaffolds woven with fibrin-small pores are represented by open bars, and scaffolds woven with fibrin-large pores are represented by cross-hatched bars.

FIG. 4D is a set of bar graphs illustrating complex shear modulus (G*) and equilibrium shear modulus (G) determined by dynamic, at ω=10 rad/sec and γₒ=0.05, and stress-relaxation shear testing, respectively. Scaffolds woven with 2% agarose-small pores are represented by grey bars, scaffolds woven with 2% agarose-large pores are represented by left-diagonal bars, scaffolds woven with 3% agarose-small pores are represented by solid black bars, scaffolds woven with 3% agarose-large pores are represented by right-diagonal bars, scaffolds woven with fibrin-small pores are represented by open bars, and scaffolds woven with fibrin-large pores are represented by cross-hatched bars.

FIGS. 5A-5D are a series of bar graphs that represent the effect of fiber reinforcement on tensile properties measured in the warp (X) and weft (Y) directions.

FIG. 5A is a set of bar graphs illustrating that small pore scaffolds show significantly higher ultimate tensile stresses in the weft direction (diagonal bars) than in the warp direction (grey bars) as compared to large pore scaffolds. Data presented are mean ±SEM, *p<0.05.

FIG. 5B is a set of bar graphs illustrating that that both small pore and large pore scaffold structures show significantly higher ultimate tensile strain in warp direction (grey bars) than in the weft direction (diagonal bars). Data presented are mean ±SEM, *p<0.05.

FIG. 5C is a set of bar graphs illustrating tangent moduli at 0% strain in warp direction (grey bars) and weft direction (diagonal bars). Data presented are mean ±SEM, *p<0.0001.

FIG. 5D is a set of bar graphs illustrating tangent moduli at 10% strain in the warp direction (grey bars) and weft direction (diagonal bars). Data presented are mean ±SEM, *p<0.0001.

### DETAILED DESCRIPTION

Tissue engineering seeks to repair or regenerate tissues of the body through combinations of implanted cells, biomaterial scaffolds, and biologically active molecules. The rapid restoration of native tissue biomechanical function remains an important challenge, emphasizing the need to replicate specific structural and mechanical properties by using novel scaffold designs. To this end, a micro-scale three-dimensional weaving technique is disclosed herein that functions to generate anisotropic three-dimensional woven structures that provide the basis for composite scaffolds and tissue constructs by consolidation with a cell-hydrogel mixture, in some embodiments. In one non-limiting example, the disclosed composite scaffolds can exhibit anisotrophic mechanical properties on the same order of magnitude of values reported for native articular cartilage, as assessed by compressive, tensile, and shear testing. The instantly disclosed subject matter provides that a cell-supporting scaffold can be engineered with initial properties that reproduce the anisotrophy, viscoelasticity, and tension-compression nonlinearity of a target tissue, including particularly native articular cartilage.

Accordingly, disclosed herein is a tissue restoration implant comprising a three-dimensional fiber scaffold for the functional tissue engineering of a target tissue, including, but not limited to, articular cartilage, that qualitatively and quantitatively mimics the behavior and mechanical properties of the target tissue without the need for extended in vitro culture. A microscale three-dimensional weaving technique is further disclosed in some embodiments, wherein a biodegradable yarn is weaved into a porous textile to yield a tissue restoration implant comprising a three-dimensional fiber scaffold.

Thus, provided herein is a tissue restoration implant comprising a three-dimensional fiber scaffold that can be used in tissue repair, restoration, and/or regeneration. The three-dimensional fiber scaffold can comprise at least three systems of fibers, wherein two of the three fiber systems define an upper layer, a lower layer and a medial layer between the upper layer and the lower layer within the three-dimensional fiber scaffold, and wherein one of the at least three fiber systems interconnects the upper layer, the lower layer and the medial layer. The at least three fiber systems can each comprise a biocompatible material, and the biocompatible material optionally comprises an absorbable material, a non-absorbable material or combinations thereof. The scaffold can provide a characteristic that functions to restore a tissue upon implantation, and representative characteristics include but are not limited to inhomogeneity, anisotropy, non-linearity, viscoelasticity, and combinations thereof.

The in-plane strength and impact performance of embodiments of the presently disclosed tissue restoration implants in tension, compression, shear, and bending are quantified. The results indicate significant increases in all measured properties of the tissue restoration implants as compared to an equivalent thickness of 2-D woven material made using standard laminating techniques.

I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” (e.g., “a PEP”) includes a plurality of such cells (e.g., a plurality of PEPs), and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The terms “inhomogeneous”, “inhomogeneity”, “heterogeneous”, “heterogeneity”, and grammatical variations thereof, are meant to refer to a scaffold and/or fiber as disclosed herein, which does not have a homogeneous composition along a given length or in a given volumetric section. In some cases an inhomogeneous tissue engineering implant as disclosed herein comprises a composite material, such as a composite comprising a three-dimensional scaffold as disclosed herein, cells of the tissue of interest, and a cell matrix that supports the cells. In another example, an inhomogeneous scaffold as disclosed herein can comprise one or more individual fiber systems which vary in fiber strength according to a predetermined profile, such as a profile associated with the tissue and or other location in a subject where the scaffold will be implanted. Such profiles can be developed based on information available in the art for a given tissue, and/or can be determined by testing techniques such as those disclosed herein and/or techniques know in the art. Thus, it is an aspect of the terms “inhomogeneous”, “inhomogeneity”, and grammatical variations thereof to encompass the control of individual fiber strengths in a scaffold.

As used herein, the terms “anisotropic”, “anisotropy”, and grammatical variations thereof, refer to properties of a scaffold and/or fiber system as disclosed herein, which can vary along a particular direction. Thus, the fiber and/or scaffold can be stronger or stiffer in one direction versus another. In some embodiments this can be accomplished by changing fibers (such as but not limited to providing fibers of different materials) in warp versus weft directions, and in the Z direction, for example. Thus, anisotropic characteristics parallel native properties of a tissue, and it is desirable to match or approximate native properties. Thus, strength can be provided in the direction needed and indeed it is possible to restore properties of a tissue almost immediately without necessarily needing for cells to grow into functional tissues. However, in some embodiments cells are provided and the growth into functional tissues is also provided. Further, in some embodiments the scaffold can comprise at least some, if not all, absorbable materials such that degradation of the scaffold occurs over time. Thus, in some embodiments, the scaffold is replaced by tissue.

In some embodiments, the terms “anisotropic”, “anisotropy” and grammatical variations thereof, can also include the provision of more fiber in a predetermined direction. This can thus include a change of diameter in a fiber over a length of the fiber, a change in diameter at each end of the fiber, and/or a change in diameter at any point or section of the fiber; includes change in cross-sectional shape of the fiber; includes change in density or number of fibers in a volumetric section of the scaffold; includes the use of monofilament fibers and or multifilament fibers in a volumetric section of the scaffold; and even includes the variation in material from fiber system to fiber system and along individual fibers in a volumetric section of the scaffold.

The terms “non-linear”, “non-linearity”, and grammatical variations thereof, refers to a characteristic provided by a scaffold and/or fiber system as disclosed herein such that the scaffold and/or fiber system varies in response to a strain. As would be appreciated by one of ordinary skill in the art after review of the present disclosure the scaffolds and/or fiber systems disclosed herein provide stress/stain profiles that mimic that observed in a target or predetermined tissue. As such stress/strain responses are typically described with reference to a plot, stress/strain responses can be referred to as “non-linear”. An important non-linear property of most biological tissues is the presence of significant differences in the strength, stiffness, and/or other properties as measured in tension in comparison to those measured in compression but along the same axis or direction.

The terms “viscoelastic”, “viscoelasticity”, and grammatical variations thereof, are meant to refer to a characteristic provided by a scaffold and/or fiber system as disclosed herein, which can vary with a time or rate of loading. It is thus envisioned that appropriately viscoelastic scaffolds and/or fiber systems provide time or rate of loading characteristics that match or approximate that observed in the predetermined tissue. This characteristic pertains to dissipation of energy, which can be provided by the scaffold itself, the scaffold as a composite with cells growing therein, and can also be accomplished in the choices of fibers that are included in the scaffold. As a particular example it can be desirable to provide a scaffold that approximates the viscoelastic properties of cartilage.

The terms “restore”, “restoration” and grammatical variations thereof refer to any qualitative or quantitative improvement in a target or predetermined tissue observed upon implantation of a scaffold as disclosed herein. Thus, these terms are not limited to full restoration of the tissue to a normal healthy function, although these terms can refer to this. Rather, these terms are meant to any level of improvement observed in the tissue.

The terms “bio-compatible” and “medically acceptable” are used synonymously herein and are meant to refer to a material that is compatible with a biological system, such as that of a subject having an tissue to be restored in accordance with the presently disclosed subject matter. Thus, the term “bio-compatible” is meant to refer to a material that can be implanted internally in a subject as described herein.

The term “absorbable” is meant to refer to a material that tends to be absorbed by a biological system into which it is implanted. Representative absorbable fiber materials include but are not limited to polyglycolic acid (PGA), polylactic acid (PLA), polyglycolide-lactide, polycaprolactone, polydioxanone, polyoxalate, a polyanhydride, a poly(phosphoester), catgut suture, collagen, silk, chitin, chitosan, hydroxyapatite, bioabsorbable calcium phosphate, hyaluronic acid, or any other medically acceptable yet absorbable fiber.

The term “non-absorbable” is meant to refer to a material that tends not to be absorbed by a biological system into which it is implanted. Representative non-absorbable fiber materials include but are not limited to polypropylene, polyester, polytetrafluoroethylene (PTFE) such as that sold under the registered trademark TEFLON® by E.I. DuPont de Nemours & Co., expanded PTFE (ePTFE), polyethylene, polyurethane, polyamide, nylon, polyetheretherketone (PEEK), polysulfone, a cellulosic, fiberglass, an acrylic, tantalum, polyvinyl alcohol, carbon, ceramic, a metal (e.g., titanium, stainless steel) or any other medically acceptable yet non-absorbable fiber.

The terms “resin”, “matrix”, or “gel” are used the art-recognized sense and refer to any natural or synthetic materials that can occupy the pore space of the fiber scaffold have characteristics suitable for use in accordance with the presently disclosed subject matter. Representative “resin”, “matrix”, or “gel” materials thus comprise bio-compatible materials.

The term “composite material”, as used herein, is meant to refer to any material comprising two or more components. One of the components of the material can optionally comprise a matrix for carrying cells, such as a gel matrix or resin.

II. Three-Dimensional Fiber Scaffold

In some embodiments, each fiber system of the fiber scaffold comprises a biocompatible material. Optionally, the biocompatible material comprises a material selected from the group including, but not limited to, an absorbable material, a non-absorbable material and combinations thereof. Further, the three-dimensional matrices can be formed of a biodegradable, non-degradable, or combination of biodegradable and non-degradable materials which have been configured to produce high cell densities by allowing adequate diffusion of nutrients and waste as well as gas exchange, while in vitro or in vivo, prior to remodeling and integration with host tissue.

Absorbable material for use in the disclosed fiber scaffold can be selected from the group including, but not limited to, polyglycolic acid (PGA), polylactic acid (PLA), polyglycolide-lactide, polycaprolactone, polydioxanone, polyoxalate, a polyanhydride, a poly(phosphoester), catgut suture, collagen, silk, chitin, chitosan, hydroxyapatite, bioabsorbable calcium phosphate, hyaluronic acid, elastin, and combinations thereof.

Non-absorbable material for use in the disclosed 3-D fiber scaffold can be selected from the group including, but not limited to, polypropylene, polyester, polytetrafluoroethylene (PTFE), expanded PTFE (ePTFE), polyethylene, polyurethane, polyamide, nylon, polyetheretherketone (PEEK), polysulfone, a cellulosic, fiberglass, an acrylic, tantalum, polyvinyl alcohol, carbon, ceramic, a metal, and combinations thereof.

The fiber scaffold can be made from biocompatible fibers, including textured fibers that provide a much lower bulk density filling than non-texturized fiber. The low bulk density of textured fibers can provide for implantation of a significant numbers of cells.

Fiber diameters can be of any suitable length in accordance with characteristics of the target or predetermined tissue in or at which the implant is to be placed. Representative size ranges include from about 25 μm to about 100 μm in diameter. As would be apparent to one in ordinary skill in the art upon review of the present disclosure, 25 μm comprises approximately the size of a microsurgery suture. In some embodiments the diameter of the fibers provides the appropriate integrity for the fiber to be held under tension and therefore implemented in a process of making as disclosed herein.

Fibers can be monofilament, multifilament, or a combination thereof, and can be of any shape or cross-section, including but not limited to bracket-shaped ([), polygonal, square, I-beam, inverted T shaped, or other suitable shape or cross-section. The cross-section can vary along the length of fiber. Fibers can also be hollow to serve as a carrier for therapeutic agents (e.g., cells, antibiotics, growth factors, etc.) as described herein. The concentration of the active agent or agents can vary linearly, exponentially or in any desired fashion. The variation can be monodirectional, that is, the content of one or more therapeutic agents decreases from the first end of the fibers or subset of the fibers to the second end of the fibers or subset of the fibers. The content can also vary in a bidirection fashion, that is, the content of the therapeutic agent or agents increases from the first ends of the fibers or subset of the fibers to a maximum and then decreases towards the second ends of the fibers or subset of the fibers.

Applicants have developed a tissue restoration implant comprising a three-dimensional fiber scaffold formed as disclosed herein that have been selected to impart a novel architecture characterized by improved anisotropic, inhomogeneous, nonlinear, and viscoelastic properties, in some embodiments. The construction of the fiber system contributes to the form and/or three-dimensional shape of the scaffold. Therefore, a new generation of scaffolds and methods of making and using the same have been provided in accordance with the presently disclosed subject matter.

The ability of articular cartilage to withstand extremely high mechanical stresses has been attributed to the complex ultrastructure and mechanical properties of the tissue. In particular, tension-compression nonlinearity, which accounts for approximately 2 orders of magnitude difference between the tensile and compressive moduli of native cartilage (Soltz, M. A., Ateshian, G. A. (2000) J. Biomech. Engng. 122:576; Cohen, B., Lai, W. M., and Mow, V. C. (1998) J Biomech Eng 120:491-496; Huang, C. Y., Stankiewicz, A., Ateshian, G. A., and Mow, V. C. (2005) J Biomech 38:799-809; Mizrahi, J. Maroudas, A., Lanir, Y., Ziv, I, and Webber, T. J. (1999) Biorheology, 23:311-330; Soulhat, J., Buschmann, M. D., and Shirazi-Adl, A. (1999) J Biomech Eng 121:340-347), is believed to play an important role in its load bearing capacity by enhancing fluid pressurization under compression (Ateshian, G. A. J Biomech Eng 119:81-86 (1997); Soltz, M. A., Ateshian, G. A., J Biomech 31:927-934 (1998)). In this respect, the design of the disclosed three-dimensional fiber scaffold can mimic the behavior of a target tissue, such as cartilage, as a fiber-reinforced composite, albeit at a larger scale (micro-scale instead of nano-scale fibers). Additional profile information for cartilage can be found in the Examples presented herein.

The presently disclosed subject matter is similarly applicable to a variety of other tissues and organs that comprise fibrous components as well as cells, and require mechanical integrity to function properly in the body. Representative characteristics of these tissues include inhomogeneity, anisotropy, nonlinearity, viscoelasticity, and combinations thereof. Representative tissues include but are not limited bone, tendon, ligament, intervertebral disc, meniscus, bladder, cardiac muscle, skeletal muscle, myocardium, fascia, adipose tissue, nerve, heart valve, intestine, lung, blood vessels, as well as organs such as kidney, liver, pancreas, stomach, and colon. The presently disclosed subject matter is also applicable to tissues and organs of the dental and craniofacial system, such as teeth, palate, calvarium, and periodontal ligament. Additionally, characteristics of interest for these and other tissues of interest can be profiled based on information available in the art for a given tissue, and/or can be determined by testing techniques such as those disclosed herein and/or techniques know in the art.

In some embodiments, the three-dimensional fiber scaffold comprises a three-dimensional textile scaffold. In this case the fiber systems are referred to as yarn systems.

In some embodiments, the three-dimensional fiber scaffold comprises three orthogonally woven fiber systems, a plurality of braided fiber systems, a plurality of circular woven fiber systems, or combinations thereof.

Thus, the presently disclosed subject matter provides in some embodiments 3-D woven fiber scaffolds for use in tissue restoration, repair, and/or regeneration. The scaffold can be used in its native form, as a composite material in combination with other materials, as an acellular (non-viable) matrix, or combined with cells and/or bioactive molecules (growth factors, for example) for use in repair, replacement, and/or regeneration of diseased or traumatized soft tissue and/or tissue engineering applications.

An advantage of the disclosed technology is the ability to produce biomaterial scaffolds and composite matrices that have precisely defined mechanical properties that can be inhomogeneous (vary with site), anisotropic (vary with direction), nonlinear (vary with strain), and viscoelastic (vary with time or rate of loading). By combining a fiber-based scaffold with a biocompatible resin or matrix, an advantage of the composite matrix is that a microenvironment of embedded cells can be controlled to promote appropriate cell growth or activity while providing for the prescribed mechanical properties. Achieving these characteristics can be facilitated using a matrix and fiber in combination.

Cartilage precursor cells, including chondrocytes, bone precursor cells, fibroblasts, and others, differ significantly from some types of cells, such as hepatocytes, in their requirements for nutrient and gas exchange. As a result, the 3-D fiber scaffolds can be suitably configured as tighter or looser structures, depending on the particular method of use, and target tissue.

The thickness and composition of the various layers, and thereby the entire three-dimensional scaffold, can be altered and customized to fit a variety of desired medical indications, as would be readily apparent to one of skill in the art after a review of the present disclosure. Thus, a fiber scaffold having more than three fiber systems is also provided in accordance with the presently disclosed subject matter, including textile scaffolds having four and five fiber systems. The additional fiber systems can comprise absorbable materials, non-absorbable materials, or combinations thereof, depending on the particular application for the scaffold.

The three-dimensional textile scaffold comprises at least three primary systems of fibers. A first system includes a plurality of x-fibers (or warp fibers) running straight and in a spaced parallel relation along the x-axis. A second system includes a plurality of y-fibers (or weft fibers) running straight and in a spaced parallel relation along the y-axis. The x-fibers and y-fibers, and thus the first and second systems, can be disposed in a mutually orthogonal relations, such that the x and y-axes are defined as in a Cartesian coordinate system.

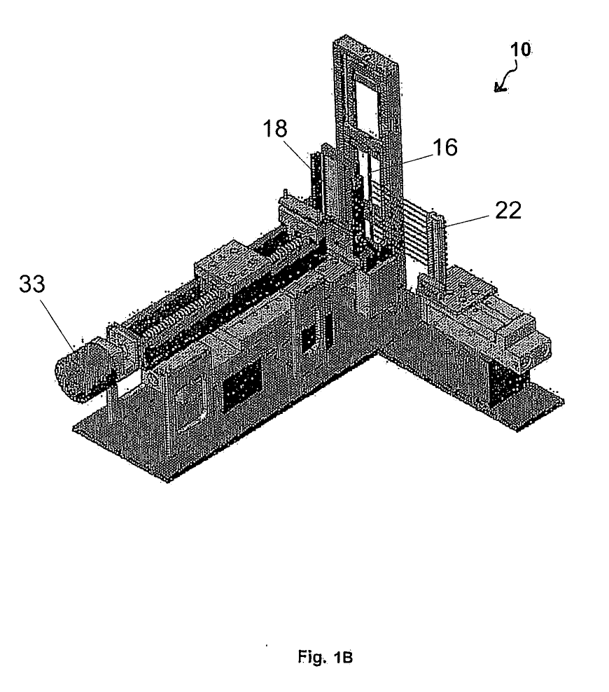

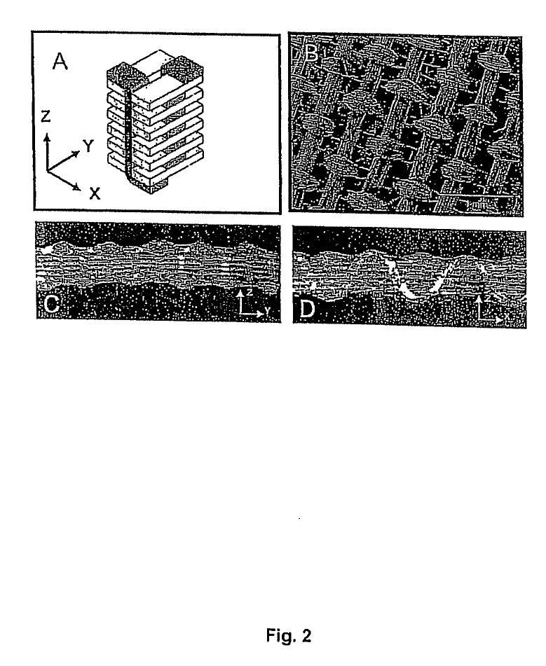

A third system includes a plurality of z-fibers running in parallel relation through the planes of x-fibers and y-fibers, such that z-fibers can be said to interconnect or bind all layers forming the three-dimensional scaffold. The z-fibers generally extend along the Cartesian z-axis such that z-fibers are mutually orthogonal to both x-fibers and y-fibers. Stated differently, the third system is disposed in an out-plane that is perpendicular to the in-plane defined by the first and second systems. See FIGS. 2A-2D.

The three fiber systems are interlaced so as to provide a plurality of pores within the textile scaffold. In some embodiments, the interstices further comprise a pore size ranging from about 10 μm to about 250 μm. In other embodiments, the interstices further comprise a pore size ranging from about 25 μm to about 175 μm. In further embodiments, the interstices further comprise a pore size ranging from about 50 μm to about 125 μm. As would be readily apparent to one of skill in the art, the dimensions of the interstices can be optimized for the particular intended use.

The scaffold can be advantageously not crimped so that interstices remain intact after the intermeshing of the at least three fiber systems. The at least three fiber systems can be secured to each other at one or more contact points to facilitate maintenance of interstices while also providing cuttability and suturability. The securing or setting of the at least three fiber systems at a contact point can be accomplished by any suitable technique, including but not limited to sonication or heat molding.

Setting of the yarn systems can be done via any of a number of art-recognized techniques, including but not limited to ultrasonication, a resin, infrared irradiation, heat or any combination thereof. Setting of the yarn systems within the scaffold in this manner provides cuttability and suturability.

Setting of the yarn can also be achieved by coating one or more surfaces of the structure with a biocompatible material using techniques such as electrospinning, electrospraying, spray coating, plasma coating, or dipping. These methods can also be used to provide desirable geometric, chemical, or physical properties to one or more surfaces of the structure. For example, electrospraying can be used to coat one surface of the structure to provide a smooth surface with nanometer scale surface roughness.

Sterilization is performed by methods such as autoclave, radiation, hydrogen peroxide, ethylene oxide, and the like, as would be readily apparent to one of ordinary skill in the art.

III. Methods of Making Three Dimensional Fiber Scaffolds

III.A. Weaving

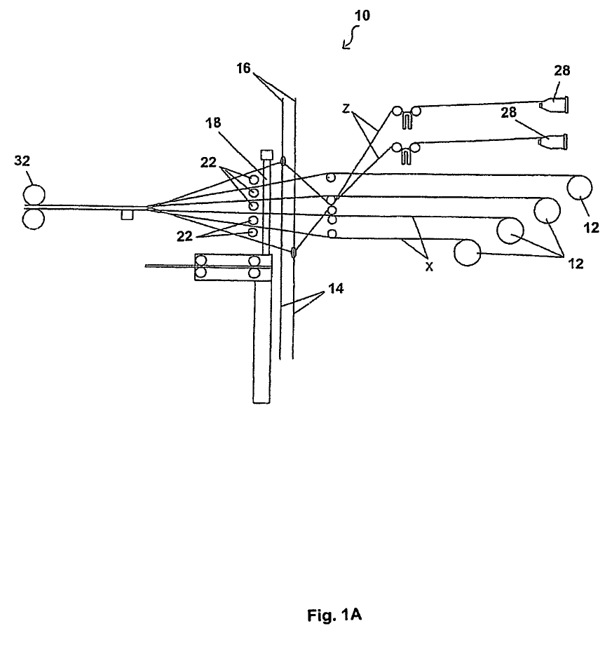

A method for producing a tissue restoration implant comprising a three-dimensional fiber scaffold is also disclosed. The method comprises forming a three-dimensional fiber scaffold with at least three fiber systems interconnecting the plurality of layers, and wherein the three dimensions of the scaffold define internal and superficial positions within the scaffold. The disclosed method can employ a weaving loom, referred to at 10 (FIGS. 1A and 1B), constructed to produce precise structures from fine diameter fibers. Weaving machine 10, which can be computer controlled, produces true three-dimensional shapes by placing fibers axially (x-warp direction), transversely (y-weft, or filling direction), and vertically (z-thickness direction).

Thus, in some embodiments, the process by which a three-dimensional fiber scaffold in accordance with the presently disclosed subject matter can be formed is further described with reference to the schematic shown in FIG. 1A and the perspective view show in FIG. 1B. In loom 10 lengthwise or x-fibers X are drawn under tension from an x-fiber feeding device 12 such as a set of warp beams (as shown) or a creel (not shown), between heddles 14 of harnesses 16, and through a beat-up reed 18, thereby forming systems of x-fibers X which are in horizontal and vertical alignment. Crosswise or y-fibers Y (not shown) are inserted between the systems of x-fibers X using fill insertion rapiers 22. In some embodiments, all y-fibers Y are inserted simultaneously in order to guarantee their straightness within the core of the 3-D fiber scaffold and to increase productivity. Beat-up reed 18 is actuated to apply force on y-fibers Y as the 3-D fiber scaffold is being formed, thereby packing x-fibers X and y-fibers Y into a structure having interstices or pores of a desired pore size. Z-fibers Z are drawn under tension from a z-fiber feeding device 28 such as a creel with bobbins (as shown) or one or more beams (not shown), and inserted through the layers formed by the systems of x-fibers X and y-fibers Y under the control of harnesses 16 with cross-moving heddles 14 and beat-up reed 18. Take-up roll 32 can be used to advance the 3-D fiber scaffold forwardly.

All operations can be computer controlled. For example, change of yarn densities can be achieved for warp by altering the reed density and warp arrangement and for weft by varying a computer program controlling the take-up speed of a stepper motor 33 (shown in FIG. 1B) operatively connected with weaving machine 10.

In some embodiments, a balloon technique is employed, whereby a small balloon placed within a hollow rapier 22 is used to insert small fibers in place, such as fibers in the Y direction in an orthogonally woven structure.

The thickness and composition of the layers of the 3-D fiber scaffold, and thereby the entire structure, can be altered and customized to fit a variety of applications. For example, additional fiber systems can be included within the upper layer, lower layer, and/or medial layer of the 3-D fiber scaffold. In some embodiments, (+)/(−) bias fibers can be incorporated within the 3-D fiber scaffold. Thus, 3-D fiber scaffolds having more than three fiber systems are provided in accordance with the presently disclosed subject matter, including scaffolds having four and five fiber systems.

In contrast to standard 2-D weaving which requires lamination of separate layers to achieve the appropriate thickness, the presently disclosed method involves in some embodiments simultaneous weaving of fibers in three orthogonal dimensions. In this design, the three-dimensional woven structure serves a load-bearing function. Thus, in some embodiments, the three-dimensional structure reinforces a hydrogel that acts to consolidate the structure and facilitate cell growth and extracellular matrix formation.

Accordingly, a tissue restoration implant adapted for use with a pre-determined tissue, comprising a three-dimensional fiber scaffold, the scaffold comprising at least three systems of fibers, wherein the fiber scaffold, or one or more of the fiber systems, provide a characteristic that functions to restore the pre-determined tissue upon implantation is disclosed. By altering one or more of the initial design variables, a composite scaffold can be designed and fabricated with initial mechanical properties that are anisotropic, nonlinear, and viscoelastic, with values of mechanical test parameters that mimic a target tissue, including as a non-limiting example native articular cartilage, even in the absence of cells and extracellular matrix. Therefore, an advantage of the presently disclosed subject matter is that every fiber can be selected individually and woven into a construct. Using this method of assembly, customized structures can be easily created by selectively placing different constituent fibers (e.g., fibers of various material composition, size, and coating/treatment) throughout the preform. In this manner, physical and mechanical properties of the scaffold can be controlled; pore sizes can be chosen, directional properties can be varied, and discreet layers can be formed. Using this technique, characteristics (e.g., inhomogeneity and anisotropy) of various tissues have been created by constructing a scaffold that mimics the normal tissue network (e.g., stratified tissue network) using a single, integral scaffold.

Representative advantages of the presently disclosed three-dimensional weaving techniques are: (1) production of true three-dimensional architecture with no lamination of multiple layers; (2) orthogonal weaving resulting in no fiber crimp; (3) complete control of multi-directional (including but not limited to anisotropic) mechanical properties; (4) complete control of fiber spacing and volume fraction in each axis; and (5) complete selection of the properties of each individual fiber in the construct.

Further, the disclosed process eliminates fiber crimp and forms a true three-dimensional structure. In general, most current three-dimensional textile composites are constructed by laminating multiple 2-D structures together and the lamination interface between multiple layers is the weak point in the composite where debonding or delamination occurs. Because the disclosed weaving method provides for no “crimping” of the in-plane fibers as in a standard woven matrix, the straightness decreases buckling of individual fibers and significantly improves their strength and stiffness properties under both compressive and tensile stresses.

The following patents and patent publications are herein incorporated by reference in their entirety: U.S. Pat. No.5,465,760 issued to Mohamed et al. on Nov. 14, 1995; U.S. Pat. No. 5,085,252 issued to Mohamed et al. on Feb. 4, 1992; published PCT international application WO01/38662, published May 31, 2001; published PCT international application WO02/07961, published Jan. 31, 2002; and published U.S. patent application US2003/0003135, published Jan. 2, 2003.

III.B. Seeding the Cells

The three-dimensional fiber scaffolds can be infiltrated with a cell-seeded gel material to form a composite construct or implant. The gel biomaterial can be one of many different types of crosslinkable, photocrosslinkable, temperature sensitive, or other gel that can sustain cell growth and provide mechanical function to the scaffold. Representative gels include, but are not limited to, fibrin, alginate, agarose, elastin, chitosan, silk, polyethylene glycol, MATTRIGEL™ gel, hyaluronic acid, and collagen. These gels can be used in native form or following modification.

Also provided is the use of a hydrogel forming material within the core of the fibers. A hydrogel is defined as a colloid in which the disperse phase (the colloid) has combined with the continuous phase (water) to produce a viscous jellylike product. (Dictionary of Chemical Terms, 4th Ed., McGraw Hill (1989)). Hydrogels are able to swell rapidly in excess water and retain large volumes of water in their swollen structures. The polymeric material comprising the hydrogel can absorb more than 20% of its weight in water, though formed hydrogels are insoluble in water and they maintain three-dimensional networks. (Amidon, Gordon L., (2000) Transport Processes in Pharmaceutical Systems, Drugs and the Pharmaceutical Sciences; v. 102 New York Marcel Dekker, Inc.,). Hydrogels are usually made of hydrophilic polymer molecules crosslinked either by chemical bonds or by other cohesion forces such as ionic interaction, hydrogen bonding, or hydrophobic interaction. (J. I. Kroschwitz, (1990) Concise Encyclopedia of Polymer Science and Engineering, New York, Wiley, XXIX, p 1341).

A representative method for combining the three-dimensional fiber scaffolds with a resin or gel matrix comprises a vacuum-assisted molding process. This technique can utilize vacuum pressure to draw the gel, while still in its liquid form, into the three-dimensional fiber scaffold, effectively filling the pore spaces and encapsulating the fibers. Once the gel has completely infused the scaffold, it is solidified by an appropriate cross-linking method, for example, to form the composite construct. When seeding cells and/or bioactive molecules into the scaffolds, they are optionally mixed into the liquid gel prior to infusion. The large, ordered, and interconnected pores of the three-dimensional scaffold allow for consistent and even distribution of cells throughout the composite implant.

The three-dimensional fiber scaffolds can be seeded with cells in some embodiments, optionally mammalian cells, such as human cells. More particularly, cells can include but are not limited to primary cells, undifferentiated progenitor cells, chondrocytes, bone-precursor cells, stem cells, synovial cells, umbilical cord cells, cord blood cells, muscle stem cells, adipose cells, preadipocytes, hematopoietic stem cells, mesenchymal stem cells, cells of the periosteum, or perichondrium tissue, stromal cells, embryonic stem cells, germ cells, and combinations of any of the foregoing. As will be understood by those of skill in the art upon reading the instant disclosure, however, the scaffolds of the present invention can be seeded with any cell type, including two or more different cell types, which exhibits attachment and ingrowth and is suitable for the intended target tissue, tissues, and/or envisioned location of implantation for the three-dimensional fiber scaffold.

Further, cells can be derived from the host, a related donor, or from established cell lines. In some embodiments of the presently disclosed subject matter, the scaffolding is constructed such that initial cell attachment and growth occur separately within the matrix for each population, for example, bone precursor and chondrocyte cell populations. Alternatively, a scaffolding, such as but not limited to a unitary scaffolding, can be formed of different materials to optimize attachment of various types of cells at specific locations. As would be apparent to one of skill in the art, attachment can be a function of both the type of cell and matrix composition.

The tissue restoration implant can further comprise a cell growth modulating material. The cell growth modulating material can be selected from a group including but not limited to growth factor, a cytokine, a chemokine, a collagen, gelatin, laminin, fibronectin, thrombin, lipids, cartilage oligomeric protein (COMP), thrombospondin, fibrin, fibrinogen, Matrix-GLA (glycine-leucine-alanine) protein, chondrocalcin, tenascin, a mineral, an RGD (arginine, glycine, aspartic acid) peptide or RGD-peptide containing molecule, elastin, hyaluronic acid, a glycosaminoglycans, a proteoglycan, water, an electrolyte solution, and combinations thereof of these molecules or their fragments. These cell modulating materials can be attached to the fibers, gel, or both, using chemical or physical modification such that they can be immobilized in a manner that allows biochemical interaction with cells, or in a manner that allows controlled release from the structure to influence cell behavior either locally or systemically. These cell modulating materials can be localized to specific regions of the structure such as individual fibers, fibers systems, segments of fibers, embedded within individual fiber materials or within hollow fibers, or within specific sites of the gel matrix such that they are delivered in a prescribed temporal and spatial pattern.

The dimensions, size, and shape of a fiber used in accordance with the presently disclosed subject matter can further be selected to regulate a rate of cell growth modulating material release. For example, an open-ended hollow fiber with a relatively large internal diameter will release a loaded cell growth modulating material at a greater rate than an identically-shaped open-ended hollow fiber with a smaller internal diameter.

In some embodiments, the cells can be cultured under standard culture conditions to expand the number of cells followed by removal of the cells from culture plates and administering into the three-dimensional scaffold prior to or after implantation of the device. Alternatively, the isolated cells can be injected directly into the three-dimensional scaffold and then cultured under conditions that promote proliferation and deposition of the appropriate biological matrix prior to in vivo implantation.

The cells can be seeded on the disclosed scaffold for a short period of time, e.g. less than one day, just prior to implantation, or cultured for longer period, e.g. greater than one day, to allow for cell proliferation and matrix synthesis within the seeded scaffold prior to implantation.

In some embodiments, a stratified construct that contains two or more distinct tissue types can be engineered by preparing a scaffold comprising functionally unique layers. This type of architecture can be formed by any suitable approach as might be apparent to one of ordinary skill in the art after a review of the present disclosure. By way of example and not limitation, such a scaffold can be formed by selectively placing pre-treated fibers (i.e. fibers treaded with biologically active agents such as but not limited to cell growth modulating materials) into discreet positions on the loom prior to weaving. Once the process begins, these layers can be woven together into one integral scaffold possessing multiple functionalities. For example, a tissue restoration implant, which integrates a first tissue layer and a second, different tissue layer within a single scaffold, can be formed by weaving fibers into lower layers of the scaffold that facilitate ingrowth of the first tissue, while the upper layers contain fibers must support the second tissue.

IV. Implantation Methods

The tissue restoration implants of the presently disclosed subject matter can be injected or implanted into any acceptable tissue, including but not limited to, cartilage, bone, tendon, ligament, intervertebral disc, meniscus, bladder, cardiac muscle, skeletal muscle, myocardium, fascia, adipose tissue, nerve, heart valve, intestine, lung, blood vessels, as well as organs such as kidney, liver, pancreas, stomach, and colon. When the tissue restoration implant is delivered to a site under circumstances where implant migration is a concern, anchoring sutures or hooks can be incorporated such that the tissue restoration implant can be attached and maintained in the desired position.

In some embodiments, the tissue restoration implant is configured and dimensioned to be mounted in both an area of damaged or destroyed tissue that has been removed, and in an adjacent healthy area of tissue. When the tissue restoration implant is placed in an area of removed tissue, communication is established between the healthy tissue and the damaged tissue area via the three-dimensional tissue scaffold, permitting vascular invasion and cellular migration. The tissue scaffold can be implanted using standard surgical methods or can be implanted using less-invasive or minimally invasive methods such as arthroscopy or laparoscopy. The scaffold can be attached in place using a variety of methods including but not limited to surgical sutures, screws, nails, tacks, glues, adhesives, or cements.

Further with respect to the disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

The instant Examples pertain to a biomimetic tissue scaffold capable of recreating the complex multiphasic behavior and material properties of a native pre-determined tissue, including particularly articular cartilage. The characteristic multiphasic behavior of the target tissue specifically includes, but is not limited to, inhomogeneity, anisotrophy, non-linearity, viscoelaticity, and combinations thereof. A microscale three-dimensional weaving technique is also disclosed for use in weaving fibers into a three-dimensional, porous textile scaffold that was infiltrated with different chondrocyte-laden hydrogels (agarose, fibrin). A series of tensile and compressive mechanical tests were performed on the composite scaffolds at time zero and during a 28 day culture period to determine their mechanical properties.

### Materials and Methods for Examples

Sample Preparation

A computer-controlled custom build loom (FIG. 1B) was used to weave the three-dimensional architecture from 100 μm diameter PGA fibers by arranging them in 3 orthogonal directions: axially (x-warp direction), transversely (y-weft, or filling direction), and vertically (z-thickness direction), yielding fiber structures with interconnected rectangular pores approximately 300 μm×300 μm×100 μm. This structure consisted of 11 total fiber layers (5 warp, 6 weft). Test samples were infused with a hydrogel matrix of either 2% agarose (Sigma-Aldrich, St. Louis, Mo., United States of America) or fibrin (Tisseel, Baxter Biosurgery, Deerfield, Ill., United States of America) using a vacuum-assisted molding process. Hydrogels were seeded with primary chondrocytes isolated from the femoral condyles of 2-3 year old skeletally mature female pigs at a density of 20×10⁶ cells/ml. Constructs were cultured at 37° C., 5% DMEM with 10% heat-inactivated fetal bovine serum, 0.1 mM non-essential amino acids, 10 mM HEPES, 100 U/ml pen/strep, and 37.5 μg/ml ascorbate-2-phosphate, with media changes every 2-3 days.

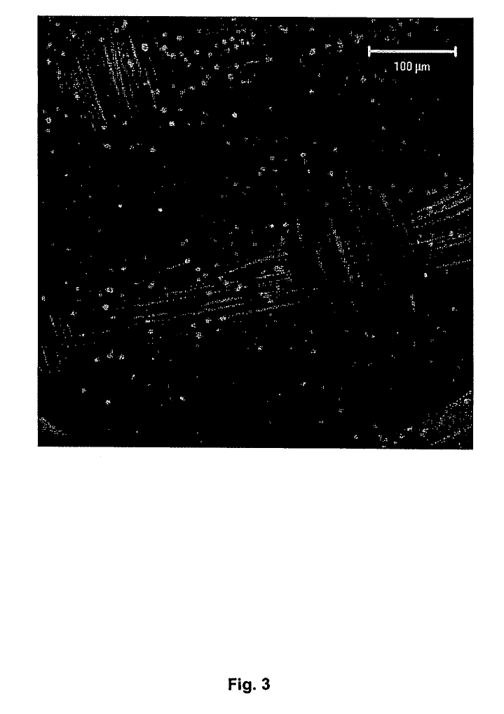

The use of hydrogel matrices maintained a rounded cell morphology (FIG. 3) to promote chondrocytic phenotype (Atala, A. et al. (1993) J Urol 150:745-747; Mauck, R. L. et al. J Biomech Eng 122:252-260 (2000); Rowley, J. A., Madlambayan, G., and Mooney, D. J. (1999) Biomaterials 20:45-53; Benya, P. D. and Shaffer, J. D. (1982) Cell 30:215-224; Watt, F. M., and Dudhia, J. (1988) Differentiation 38:140-147; Hoemann, C. D., Sun, J., Legare, A., McKee, M. D., and Buschmann, M. D. (2005) Osteoarthritis Cartilage 13:318-329; Lee, D. A., Bader, D. L. (1997) J Orthop Res 15:181-188).

Mechanical Testing

Tensile tests were performed at day 0 to determine the ultimate tensile stress, ultimate tensile strain, tangent modulus (at ε=0.1), and energy-to-failure of the composite scaffolds in two (warp and weft) independent directions. For tensile tests, dog-bone shaped test strips were uniaxially pulled until failure at 0.4 mm/s. Compressive properties were determined using a confined compression creep test on 5 mm disks at a compressive load of 10 g for 1200 s. Statistical analyses were performed by ANOVA and Fisher's PLSD (α=0.05).

Three-Dimensional Weaving and Composite Scaffold Preparation

Polyglycolic acid (PGA) yarn was used in combination with two different 3-D woven structures (with differing degrees of fiber reinforcement). Two different hydrogels were used, agarose and fibrin. These initial designs represent proof of concept of the goals of this work, but for additional applications, any combination of yarns, weaves and hydrogels can be used to produce inhomogeneity and anisotropy in a controlled manner.

The basis of the composite technology implemented herein is a three-dimensional weave of fibers in three orthogonal directions (FIGS. 2A-2B). In contrast to standard weaving methods, the disclosed process can eliminate fiber crimp and can form a true three-dimensional structure. Additional advantages include control of multi-directional (anisotropic) mechanical properties, control of fiber spacing and volume fraction in each axis, and ability to select each individual fiber in the construct.

Three-dimensional fabric structures were produced using 104 μm diameter continuous multi-filament PGA yarn (Biomedical Structures, LLC, Slatersville, R.I., United States of America). The yarn was woven into two different three-dimensional structures containing 11 total in-plane fiber layers; 5 layers were oriented in the warp direction (0° or lengthwise in the loom) and 6 layers were oriented in the weft direction (90° to the lengthwise fibers). FIGS. 2A-2D show a schematic of the three-dimensional woven scaffold and photomicrographs in the X-Y, Y-Z, and X-Z planes.

The first structure contained 24 yarns per centimeter in each of the 5 warp layers, 20 yarns per centimeter in each of the weft layers, and 24 fibers per centimeter in the Z-direction. The resulting “small pore” scaffold contained rectangular pores with dimensions of approximately 390 μm×320 μm×104 μm and a void volume of approximately 70%. Similar to the first, the second structure was woven with 24 yarns per centimeter in each of the 5 warp layers and 24 yarns per centimeter in the z-direction, but contained only 15 yarns per centimeter in each of the weft layers. The pore dimensions of this “large pore” structure measured approximately 450 μm×320 μm×104 μm and the total void volume fraction was approximately 74%.

Test samples were cut from three-dimensional woven structures and used to generate either a composite scaffold by consolidation with a biocompatible hydrogel or a composite construct by consolidation with a chondrocyte-hydrogel mixture. Typically used hydrogels agarose (2% or 3% w/v) and fibrin (100-130 mg/ml, Tissell™, Baxter Biosurgery, Westlake Village, Calif., United States of America) were evaluated. Composite scaffolds and constructs were formed by infusing the hydrogel (with or without cells) into the woven structures using a modified vacuum-assisted molding process. Using this technique, scaffolds were readily seeded with a spatially uniform distribution of cells (FIGS. 2A-2D). However, for this study, tests were carried out on composite scaffolds without cells to determine their initial mechanical properties.

Evaluation of Compressive Properties

Creep experiments were performed in a confined-compression configuration (Mow, V. C., et al. (1980) J. Biomech. Engng. 102:73), using an ELF 3200 Series precision controlled material testing system (Bose Corp., Minnetonka, Minn., United States of America). Prior to testing, sample thickness (h) was measured optically using a digital video camera (Model XDC-X700, Sony Electronics, Park Ridge, N.J., United States of America). Cylindrical test specimens were placed in a 5 mm diameter confining chamber and compressive loads were applied using a solid piston against a rigid porous platen (porosity=50%, pore size=50-100 μm). Following equilibration of a 4 gf tare load, a step compressive load of 10 gf was applied to the sample and allowed to equilibrate for 600 s. The compressive modulus (H_{A}) and hydraulic permeability (k) were determined numerically by matching the solution for axial strain (ε_{z}) to the experimental data for all creep tests using a two-parameter, nonlinear least-squares regression procedure (Cohen, B., Lai, W. M., and Mow, V. C. (1998) J Biomech Eng 120:491-496; Elliott, D. M., Guilak, F., Vail, T. P., Wan J. Y., and Setton, L. A. (1999) J Orthop Res 17:503-508) using a high-capacity materials testing system (SmartTest Series, Bose Corp., Minnetonka, Minn., United States of America).

For unconfirmed compression, strains of ε=0.04, 0.08, 0.12, and 0.16 were applied to the specimens after equilibration of a 2% tare strain. Strain steps were held constant for 900 s allows the scaffolds to relax to an equilibrium level. Young's modulus was determined by performing linear regression on the resulting equilibrium stress-strain plot.

Evaluation of Tensile Properties

Tensile experiments were performed on the composite constructs in a uniaxial configuration as described previously for cartilage (Elliot, D. M., Guilak, F., Vail, T. P., Wang, J. Y. and Setton, L. A. (1999) J Orthop Res 17:503-508; Guilak, F., Ratcliffe, A., Lane, N., Rosenwasser, M. P. and Mow, V. C. (1994) J Orthop Res 12:474-484). The protocol allowed for determination of the ultimate tensile strength, ultimate tensile strain, tensile modulus, energy to failure, and Poisson's ratio, v, of the constructs in two (X-warp and Y-weft) independent directions. After equilibration under a tare load of 10 N for 300 s, the undeformed sample length (Lₒ) was recorded as the jaw-to-jaw position.

From this point, failure tests were performed at a slow elongation rate of 0.04 mm/s in an attempt to minimize viscoelastic effects. The resulting force was recorded by the load cell and digital data acquisition system and divided by the cross-sectional area (A) for calculation of the tensile stress (σ=F/A). A tangent modulus was calculated for both the toe (E₀: ε=0) and in the linear region (E: ε=0.1) of the resulting stress-strain curve. During testing, sequential images were recorded using the automated digital video acquisition system. The images were used for measuring the local reference dimensions for strain calculations and subsequent determination of Poisson's ratio.

Evaluation of Shear Properties

Dynamic and stress relaxation shear tests of the composite constructs were performed in a PBS bath at room temperature using an ARES Rheometrics System (Rheometric Scientific, Piscataway, N.J., United States of America). Initially, a series of shear stress, relaxation tests were performed, as described previously (LeRoux, M. A., Guilak, F. and Setton, L. A. (1999) J Biomed Mater Res 47:46-53; LeRoux, M. A., et al. (2000) J of Orthop Res 18:383-392; Zhu, W., Mow, V. C., Koob, T. J., and Eyre, D. R. (1993) J Orthop Res 11:771-781). Three magnitudes of sheer strain (γ=0.001, 0.0014, and 0.0018) were applied to the sample followed by a 600 s stress relaxation period. Also, a dynamic frequency sweep was performed by prescribing a sinusoidal shear strain profile, γ=γₒ sin (ωt) at an amplitude γₒ of 0.05 and an angular frequency, ω, from 1 to 100 rad/s.

Statistical Analyses

Two-factor analysis of variance (ANOVA) tests were performed to compare the different scaffold parameters (pore size and gel type) for compressive and shear biomechanical tests. Statistical significance for tensile testing, which introduced direction (warp and weft) as a third parameter, was assessed using three-factor ANOVA. Statistical significance was reported at the 95% confidence level (p<0.05) for all tests.

### Example 1

### Characterization of the Compressive Properties of a Tissue Restoration Implant Comprising a Three-Dimensional Fiber Reinforcement

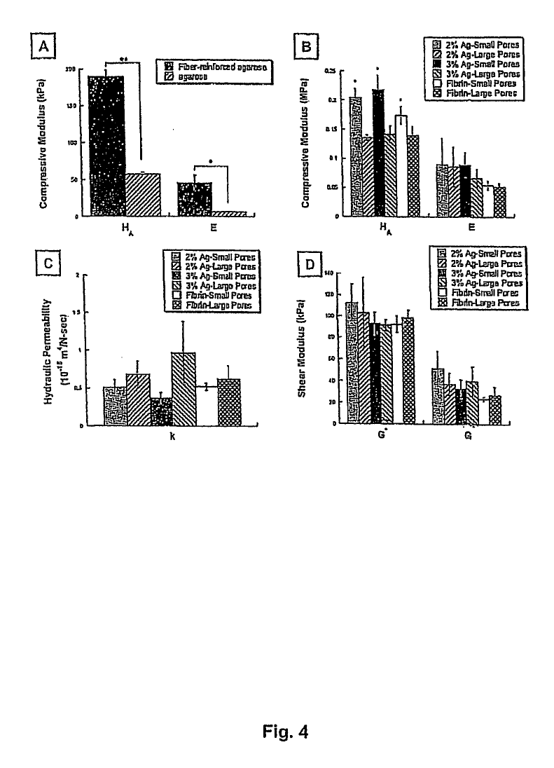

The addition of three-dimensional fiber reinforcement increased the aggregate modulus by 4-fold and the Young's modulus by 15-fold for composite fiber scaffolds and 2% agarose gel (FIG. 4A, p<0.005). Scaffolds woven with small pores showed significantly higher aggregate moduli than those woven with large pores under confined compression (FIG. 4B, p<0.005). The mean values of H_{A} for the small and large pore scaffolds were 0.199±0.018 MPa and 0.138±0.011 MPa, respectively (mean±SEM).

Similar trends were observed in unconfined compression where mean values for Young's modulus were 0.077±0.024 MPa for small pore scaffolds and 0.068±0.018 MPa for large pore scaffolds (FIG. 4B). However, for a given woven structure the type of hydrogel (2% agarose, 3% agarose or 100-300 mg/ml fibrin) did not have any significant effect on compressive properties (FIG. 4B). Therefore, three-dimensional fiber reinforcement provided for several orders of magnitude increase in compressive properties (FIG. 4A).

Resistance to compressive loading, however, was predominantly due to inter- and intra-fiber friction among the constituent multi-filament yarns within the weave. Even though the hydrogel component appeared to be primarily responsible for the observed viscoelastic creep behavior, changes in hydrogel composition did not contribute significantly to the compressive properties of the composite scaffolds (FIG. 4B).

The apparent hydraulic permeability of the structure, as measured by confined compression creep, was similar to that of native cartilage (approximately 10⁻¹⁵ m⁴/N-s), further indicating the biomimetic properties of the composite scaffolds. Hydraulic permeability of the composite scaffolds showed no significant dependence on either the type of woven structure or the type of hydrogel (FIG. 4C).

### Example 2

### Characterization of the Tensile Properties of Three-Dimensional Fiber Reinforcement

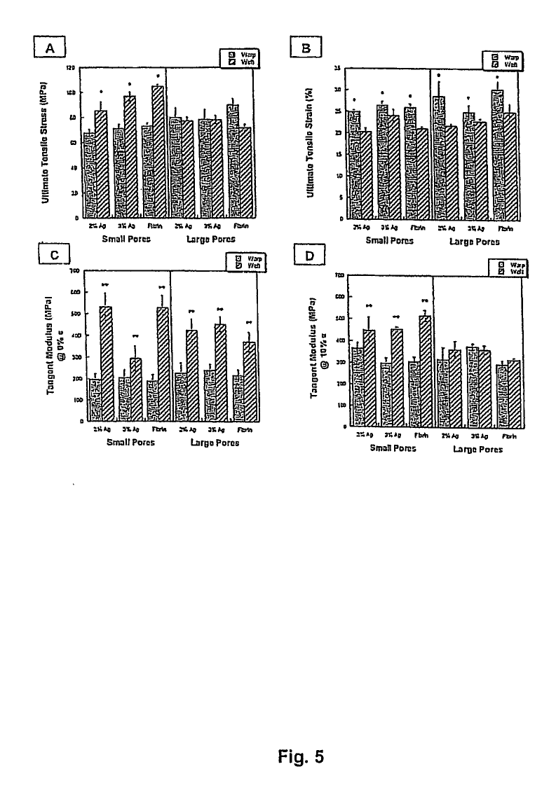

Tensile failure testing of small pore scaffolds showed significant directional dependence for values of ultimate tensile stress, ultimate tensile strain, and tensile moduli at 0%, and 10% strain levels (FIGS. 5A-D, respectively). Small pore scaffolds exhibited approximately 35% higher ultimate tensile stress when tested in the weft direction than in the warp direction (FIG. 5A, p<0.05), a finding that did not apply to large pore scaffolds. Tensile moduli calculated at 0% strain (E₀) for all scaffolds were significantly higher when tested in the weft direction than in the warp direction (FIG. 5C, p<0.0001). However, only small pore scaffolds showed significantly higher tensile moduli at 10% strain (E) when tested in the weft direction as opposed to the warp direction (FIG. 5D, p<0.0001). Values of E₀ were higher by up to approximately 250% in the weft as in the warp direction, whereas values for E were only approximately 25% higher (FIG. 5C vs. 5D). On average, tensile moduli were three orders of magnitude higher than compressive moduli. Ultimate tensile strains of all scaffolds were shown to be higher by approximately 20% in the warp direction than in the weft direction (FIG. 5B, p<0.05).

Therefore, in tension, the fiber scaffolds provided high strength and stiffness, which significantly exceeded the properties of native articular cartilage through numerous highly aligned and strong fibers oriented in the direction of the applied load.

Skeletally mature articular cartilage exhibits significant anisotropy in tension relative to the preferred orientation of collagen fibers in the surface zone, or local “split-line” direction (Guilak, F., Setton, L. A., and Kraus, V. B. (2000) In Principles of Practice of Orthopaedic Sports Medicine (ed. K. P. S. W. E. Garrett Jr., and D. T. Kirkendall) pp. 53-73 (Lippincott Williams and Wilkins, Philadelphia; Woo, S. L., et al. (1986) J. Biomech. 12:437, 1979; Akizuki, S. et al. J Orthop Res 4:379-392; Kempson, G. E., et al. (1976) Biochim Biophys Acta 428:741-760; Below, S., Arnoczky, S. P., Dodds, J., Kooima, C., and Walter, N. (1999) Arthroscopy 18:613-617 (1999)). For example, the tensile failure stress of native cartilage tissue tested parallel to the split-line direction has been shown to be twice as high as when tested perpendicularly to that direction (Kempson, G. E., et al. Biochim Biophys Acta 428:741-760 (1976).

The small pore scaffolds developed in this study were designed to have similar in-plane directional dependence of tensile mechanical properties. In particular, elevated magnitudes of ultimate tensile strength and tensile moduli were achieved in the weft direction of the small pore scaffolds (FIGS. 5A, 5C, 5D) by forming a biased woven structure that contained a higher fiber volume fraction in the weft direction than in the warp direction (FIG. 2A). This anisotropy, however, was not observed in the large pore scaffolds that were purposely woven with more balanced warp-weft fiber volume fractions (i.e., lower yarn density in the weft direction). Alternatively, controlled anisotrophy independent of the pore size or fiber packing density was achieved by using fibers with different sizes or chemical compositions in any of the orthogonal directions.

In addition to controlled anisotropy stemming from user-defined weaving parameters, the directional dependence in the tensile stress-strain behavior of the composite scaffolds can also be attributed to their unique three-dimensional fiber architecture, which included layers of straight fibers stacked in the alternating warp and weft directions (FIG. 2A). These orthogonally oriented layers were bound together by an interwoven set of continuous “Z-fibers” that passed in a quasi-sinusoidal path through the thickness of the fabric, in-line with the warp direction fibers in the X-Z plane (FIG. 2D). When the scaffold was pulled in the warp direction during tensile testing, the warp fibers immediately began to support the applied load and resist the axial deformation. As loading continued, the warp fibers stretched and the Z-fibers straightened and were recruited to assist in supporting the increasing load. It is this structural characteristic of the three-dimensional woven scaffold that gave rise to the higher tensile moduli in the warp direction at 10% strain than at 0% strain (FIGS. 5C-5D).

### Example 3

### Characterization of the Shear Properties of Three-Dimensional Fiber Reinforcement

No significant differences in complex shear modulus or relaxation modulus were observed with respect to the type of woven structure or the type of hydrogel. Mean values of G* and G for all scaffolds were 98.44±13.35 KPa and 35.38±9.61 KPa, respectively (FIG. 4D). The average loss angle (phase angle between stress and strain) of all tested scaffolds was 35.62±1.39 degrees (Table 1).

### EXAMPLES SUMMARY

The tissue restoration implant comprising a three-dimensional woven composite scaffold showed significant anisotropic, nonlinear, and viscoelastic properties similar to those of native articular cartilage. Overall, the inclusion of three-dimensional fiber reinforcement to the various hydrogels resulted in multiple fold increases in mechanical properties, particularly in compression (FIG. 4A). As expected, anisotropic design features in the woven scaffolds resulted in anisotroic biomechanical properties in tension (FIGS. 5A-5D). Significant effects of certain variables such as scaffold pore size were observed in specific testing configurations, but not others, as detailed hereinabove. Biomechanical properties of composite scaffolds are summarized and compared to native articular cartilage in Table 1.

The three-dimensional weaving technology allowed for the creation of a biocompatible fiber reinforcing structure that, when coupled with a cell-supporting hydrogel, formed a tissue-engineering scaffold capable of mimicking the highly complex physical and mechanical behavior of native articular cartilage. The large number of variables in this design (selection of approximately 400 individual fibers, fiber density, and packing in all directions, and gel biomaterials) provide a wide range of possible mechanical properties for tissue engineered scaffolds.

A 35% higher failure stress was observed in the weft direction than in the warp direction. Similarly, the tangent modulus was 471±15.5 MPa in the weft direction versus 321.1±14.8 MPa in the warp direction (p<0.05). The average failure strain was 0.206±0.006 in the weft direction and 0.254±0.006 in the warp direction. Scaffolds displayed approximately three orders of magnitude difference in tensile and compressive moduli, with aggregate moduli of 0.204±0.015 MPa for 2% agarose/PGA constructs at day 0. Gel type was shown to have no significant influence on mechanical behavior of scaffolds tested at day 0. After 14 days in culture, acellular scaffolds showed a 16% decrease in aggregate modulus. Furthermore, an additional 81% decrease was observed after 28 days in culture. Cell-loaded scaffolds were 34% stiffer as compared to acellular controls after 28 days (p<0.005).

TABLE 1
Table 1. Biomechanical properties of composite scaffolds are compared to
native articular cartilage. Ranges given for the composite scaffolds
include all experimental groups (i.e., two types of woven
structures and 3 types of hydrogels).

| | Composite Scaffold | Articular Cartilage |
|---|---|---|
| Tensile Properties | | |
| Ultimate tensile stress | 75-85 MPa | 15-35 MPa^{a,b} |
| Ultimate tensile strain | 22-27% | 10-40%^{a,b} |
| Tensile modulus (10% ε) | 325-400 MPa | 5-25.5 MPa^{c,d,e} |
| Poisson's ratio | 0.073-0.076 | 0.9-2.2^{f} |
| Equilibrium relaxation | 150-200 MPa | 6.5-45 MPa^{g,h} |
| modulus | | |
| Compressive properties | | |
| Aggregate modulus | 0.14-0.2 MPa | 0.1-2.0 MPaⁱ |
| Hydraulic permeability | 0.4-1.0 × 10⁻¹⁵ m⁴/ | 0.5-5.0 × 10⁻¹⁵ m⁴/ |
| | N-s | N-s^{j,k} |
| Young's modulus | 0.005-.01 MPa | 0.4-0.8 MPa^{l,m} |
| Shear properties | | |
| Equilibrium shear modulus | 0.03-0.05 MPa | 0.05-0.25 MPa^{n,o} |
| Complex shear modulus | 0.09-0.11 MPa | 0.2-2.0 MPaⁿ |
| Loss angle | ˜35° | ˜10°ⁿ |

^{a}Kempson, G. E., et al. Biochim Biophys Acta 428: 741-760 (1976).
^{b}Bader, D. L., Kempson, G. E., Barrett, A. J. and Webb, W. Biochim Biophys Acta 677: 103-108 (1981).
^{c}Akizuki, S. et al. J Orthop Res 4: 379-392 (1986).
^{d}Elliott, D. M., Guilak, F., Vail, T. P., Wang, J. Y., and Setton, L. A., J Orthop Res 17: 503-508 (1999).
^{e}Setton, L. A., Mow, V. C., Muller, F. J., Pita, J. C., and Howell, D. S. J Orthop Res 12: 451-463 (1994)
^{f}Elliott, D. M., Narmoneva, D. A., and Setton, L. A. J Biomech Eng 124: 223-228 (2002).
^{g}Huang, C. Y., Mow, V. C., and Ateshian, G. A. J Biomech Eng 123: 410-417 (2001).
^{h}Huang, C. Y., Soltz, M. A., Kopacz, M., Mow, V. C. and Ateshian, G. A. J Biomech Eng 125: 84-93 (2003).
ⁱMow, V. C. and Guo, X. E. Annu Rev Biomed Eng 4: 175-209 (2002).
^{j}Athanasiou, K., Rosenwasser, M. P., Buckwalter, J. A., Malinin, T. I., and Mow, V. C. J Orthop Res 9: 330-340 (1991).
^{k}Setton, L. A., Zhu, W. and Mow, V. C. J Biomech 26: 581-592 (1993).
^{l}Athanasiou, K. A., Agarwal, A., and Dzida, F. J. J Orthop Res 12: 340-349 (1994).
^{m}Jurvelin, J. S., Buschmann, M. D., and Hunziker, E. B. J Biomech 30: 235-240 (1997).
ⁿZhu, W., Mow, V. C., Koob, T. J., and Eyre, D. R. J Orthop Res 11: 771-781 (1993).
^{o}Setton, L. A., Mow, V. C., and Howell, D. S. J Orthop Res 13: 473-482 (1995).

### REFERENCES

The references listed below as well as all references cited in the specification are incorporated herein by reference to the extent that they supplement, explain, provide a background for, or teach methodology, techniques and/or compositions employed herein.
Akizuki, S. et al. (1986) J Orthop Res 4:379-392.
Atala, A. et al. J Urol (1993) 150:745-747 (1993).
Ateshian, G. A. J Biomech Eng (1997) 119:81-86.
Athanasiou, K. A., Agarwal, A., and Dzida, F. J. (1994) J Orthop Res 12:340-349.
Athanasiou, K., Rosenwasser, M. P., Buckwalter, J. A., Malinin, T. I., and Mow, V. C. (1991) J Orthop Res 9:330-340.
Bader, D. L., Kempson, G. E., Barrett, A. J. and Webb, W. (1981) Biochim Biophys Acta 677:103-108.
Below, S., Arnoczky, S. P., Dodds, J., Kooima, C., and Walter, N. (1999) Arthroscopy 18:613-617.
Benya, P. D. and Shaffer, J. D. (1982) Cell 30:215-224.
Buschmann, M. D., Gluzband, Y. A., Grodzinsky, A. J., Kimura, J. H., and Hunziker, E. B. (1992) J Orthop Res 10:745-758.
Cohen, B., Lai, W. M., and Mow, V. C. (1998) J Biomech Eng 120:491-496.
Elliott, D. M., Guilak, F., Vail, T. P., Wan J. Y., and Setton, L. A. (1999) J Orthop Res 17:503-508.
Elliott, D. M., Narmoneva, D. A., and Setton, L. A. (2002) J Biomech Eng 124:223-228.
Guilak, F., Ratcliffe, A., Lane, N., Rosenwasser, M. P., and Mow, V. C. (1994) J Orthop Res 12:474-484.
Guilak, F., Setton, L. A., and Kraus, V. B. (2000) In Principles of Practice of Orthopaedic Sports Medicine (ed. K. P. S. W. E. Garrett Jr., and D. T. Kirkendall) 53-73 (Lippincott Williams and Wilkins, Philadelphia).
Hoemann, C. D., Sun, J., Legare, A., McKee, M. D., and Buschmann, M. D. (2005) Osteoarthritis Cartilage 13:318-329.
Huang, C. Y., Mow, V. C., and Ateshian, G. A. (2001) J Biomech Eng 123:410-417.
Huang, C. Y., Soltz, M. A., Kopacz, M., Mow, V. C. and Ateshian, G. A. (2003) J Biomech Eng 125:84-93.
Huang, C. Y., Stankiewicz, A., Ateshian, G. A., and Mow, V. C. (2005) J Biomech 38:799-809.
Jurvelin, J. S., Buschmann, M. D., and Hunziker, E. B. (1997) J Biomech 30:235-240.
Kempson, G. E., et al. (1976) Biochim Biophys Acta 428:741-760.
Kisiday, J. et al. (2002) Proc Natl Acad Sci USA 99:9996-10001.
Lee, D. A., Bader, D. L. (1997) J Orthop Res 15:181-188.
LeRoux, M. A., et al. (2000) J of Orthop Res 18:383-392.
LeRoux, M. A., Guilak, F. and Setton, L. A. (1999) J Biomed Mater Res 47:46-53.
Mauck, R. L. et al. (2000) J Biomech Eng 122:252-260.
Mizrahi, J. Maroudas, A., Lanir, Y., Ziv, I, and Webber, T. J. (1999) Biorheology, 23:311-330.
Mohamed, M. H., Bogdanovich, A. E., Dickinson, L. C., Singletary, J. N., and Lienhart, R. B. (2001) Sampe Journal 37:8-17.
Mow, V. C. and Guo, X. E. (2002) Annu Rev Biomed Eng 4:175-209.
Mow, V. C., et al. (1980) J. Biomech. Engng. 102:73-84.
Mow, V. C., Ratcliffe, A., and Poole, A. R. (1992) Biomaterials 13:67-97.
Pei, M., et al. (2002) Faseb J 16:1691-1694.
Rowley, J. A., Madlambayan, G., and Mooney, D. J. (1999) Biomaterials 20:45-53.
Setton, L. A., Mow, V. C., and Howell, D. S. (1995) J Orthop Res 13:473-482.
Setton, L. A., Mow, V. C., Muller, F. J., Pita, J. C., and Howell, D. S. (1994) J Orthop Res 12:451-463.
Setton, L. A., Zhu, W. and Mow, V. C. (1993) J Biomech 26:581-592.
Smidsrod, O. and Skjak-Braek, G. (1990) Trends Biotechnol 8:71-78.
Soltz, M. A., Ateshian, G. A., (1998) J Biomech 31:927-934.
Soltz, M. A. and Ateshian, G. A. (1980) J. Biomech. Engng. 122:576-586.
Soulhat, J., Buschmann, M. D., and Shirazi-Adl, A. (1999) J Biomech Eng 121:340-347.
Watt, F. M., and Dudhia, J. (1988) Differentiation 38:140-147.
Woo, S. L., et al. (1979) J. Biomech. 12:437-446.
Zhu, W., Mow, V. C., Koob, T. J., and Eyre, D. R. (1993) J Orthop Res 11:771-781.
U.S. Patent Application US2003/0003135.
U.S. Pat. No. 5,465,760 Mohamed et al.
U.S. Pat. No. 5,085,252 Mohamed et al.
PCT International Application WO01/38662.
PCT International Application WO02/07961.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A tissue restoration implant adapted for use with a pre-determined tissue, comprising:
a three-dimensional fiber scaffold, the scaffold comprising at least three systems of fibers;
wherein two of the three fiber systems define an upper layer, a lower layer and a medial layer between the upper layer and the lower layer within the three-dimensional fiber scaffold;
wherein one of the at least three fiber systems interconnects the upper layer, the lower layer and the medial layer;
wherein the at least three fiber systems each comprise a bio-compatible material; and
wherein the fiber scaffold, or one or more of the fiber systems, provide a characteristic that functions to restore the pre-determined tissue upon implantation.

2. A tissue restoration implant adapted for use with a pre-determined tissue, comprising:
a three-dimensional fiber scaffold, the scaffold comprising at least three systems of fibers;
wherein two of the three fiber systems define an upper layer, a lower layer and a medial layer between the upper layer and the lower layer within the three-dimensional fiber scaffold;
wherein one of the at least three fiber systems interconnects the upper layer, the lower layer and the medial layer;
wherein the at least three fiber systems each comprise a bio-compatible material;
wherein the fiber scaffold, or one or more of the fiber systems, provide a characteristic that functions to restore the pre-determined tissue upon implantation; and
one or more cells that can develop into the pre-determined tissue.

3. The tissue restoration implant of claim 1 or claim 2, wherein the biocompatible material comprises a material selected from the group consisting of an absorbable material, a non-absorbable material and combinations thereof.

4. The tissue restoration implant of claim 3, wherein the non-absorbable material is selected from the group including but not limited to polypropylene, polyester, polytetrafluoroethylene (PTFE), expanded PTFE (ePTFE), polyethylene, polyurethane, polyamide, nylon, polyetheretherketone (PEEK), polysulfone, a cellulosic, fiberglass, an acrylic, tantalum, polyvinyl alcohol, carbon, ceramic, a metal, and combinations thereof.

5. The tissue restoration implant of claim 3, wherein the absorbable material is selected from the group including but not limited to polyglycolic acid (PGA), polylactic acid (PLA), polyglycolide-lactide, polycaprolactone, polydioxanone, polyoxalate, a polyanhydride, a poly(phosphoester), catgut suture, collagen, silk, chitin, chitosan, hydroxyapatite, bioabsorbable calcium phosphate, hyaluronic acid, elastin, and combinations thereof.

6. The tissue restoration implant of claim 1 or claim 2, wherein the fiber systems further comprise a monofilament fiber, a multifilament fiber, a hollow fiber, a fiber having a variable cross-section along its length, or a combination thereof.

7. The tissue restoration implant of claim 1 or claim 2, wherein the at least three fiber systems in at least one of the upper, medial and lower layers define a plurality of interstices within the fiber scaffold.

8. The tissue restoration implant of claim 7, wherein the interstices further comprise a pore size ranging from about 10 μm to about 250 μm.

9. The tissue restoration implant of claim 8, wherein the interstices further comprise a pore size ranging from about 25 μm to about 175 μm.

10. The tissue restoration implant of claim 9, wherein the interstices further comprise a pore size ranging from about 50 μm to about 125 μm.

11. The tissue restoration implant of claim 1 or claim 2, wherein the characteristic that functions to restore the pre-determined tissue upon implantation is selected from the group consisting of inhomogeneity, anisotropy, nonlinearity, viscoelasticity, and combinations thereof.

12. The tissue restoration implant of claim 2, wherein the one or more cells are present in a matrix.

13. The tissue restoration implant of claim 12, wherein the matrix comprises a gel.

14. The tissue restoration implant of claim 2, wherein the one or more cells are selected from the group consisting of primary cells, undifferentiated progenitor cells, chondrocytes, bone-precursor cells, stem cells, cells of the periosteum, or perichondrium tissue, and combinations thereof.

15. The tissue restoration implant of claim 1 or claim 2, wherein the pre-determined tissue is articular cartilage.

16. The tissue restoration implant of claim 1 or claim 2, comprising a cell growth modulating material.

17. The tissue restoration implant of claim 16, wherein the cell growth modulating material is selected from the group consisting of a growth factor, a cytokine, a chemokine, a collagen, gelatin, laminin, fibronectin, thrombin, lipids, cartilage oligomeric protein (COMP), thrombospondin, fibrin, fibrinogen, Matrix-GLA (glycine-leucine-alanine) protein, chondrocalcin, tenascin, a mineral, an RGD (Arginine-Glycine-Aspartic Acid) peptide or RGD-peptide containing molecule, elastin, hyaluronic acid, a glycosaminoglycan, a proteoglycan, water, an electrolyte solution, and combinations thereof.

18. The tissue restoration implant of claim 1 or claim 2, wherein the three-dimensional fiber scaffold comprises three orthogonally woven fiber systems, a plurality of braided fiber systems, a plurality of circular woven fiber systems, or combinations thereof.

19. A method of producing a tissue restoration implant for use in tissue restoration, the method comprising:
forming a three-dimensional fiber scaffold with at least three fiber systems such that two of the three fiber systems define an upper layer, a lower layer and a medial layer between the upper layer and the lower layer within the three-dimensional fiber scaffold,
wherein one of the at least three fiber systems interconnects the upper layer, the lower layer and the medial layer,
wherein the at least three fiber systems each comprise a bio-compatible material, and
wherein the fiber scaffold, or one or more of the fiber systems, provide a characteristic that functions to restore the pre-determined tissue upon implantation, whereby a tissue restoration implant is produced.

20. The method of claim 19, wherein the three-dimensional fiber scaffold comprises three orthogonally woven fiber systems, a plurality of braided fiber systems, a plurality of circular woven fiber systems, or combinations thereof.

21. The method of claim 20, wherein one of the three orthogonally woven fibers systems is inserted into the scaffold as a single fiber and severed at a pre-determined point.

22. The method of claim 19, wherein the characteristic that functions to restore the pre-determined tissue upon implantation is selected from the group consisting of inhomogeneity, anisotropy, nonlinearity, viscoelasticity, and combinations thereof.

23. The method of claim 19, wherein the pre-determined tissue is articular cartilage.

24. The method of claim 19, comprising providing in the scaffold one or more cells that can develop into a pre-determined tissue.

25. The method of claim 24, wherein the one or more cells are provided in a matrix.

26. The method of claim 25, wherein the matrix comprises a gel.

27. The method of claim 24, wherein the one or more cells are selected from the group consisting of primary cells, undifferentiated progenitor cells, chondrocytes, bone-precursor cells, stem cells, cells of the periosteum, or perichondrium tissue, and combinations thereof.

28. A method of restoring a tissue in a subject, the method comprising:
(a) providing a three-dimensional fiber scaffold formed of at least three systems of fibers,
wherein two of the three fiber systems define an upper layer, a lower layer and a medial layer between the upper layer and the lower layer within the three-dimensional fiber scaffold,
wherein one of the at least three fiber systems interconnects the upper layer, the lower layer and the medial layer,
wherein the at least three fiber systems each comprise a bio-compatible material, and
wherein the fiber scaffold, or one or more of the fiber systems, provide a characteristic that functions to restore the pre-determined tissue upon implantation; and
(b) implanting at a pre-determined site in the subject the three-dimensional fiber scaffold provided in step (a) to thereby restore a tissue in the subject.

29. The method of claim 28, wherein the characteristic that functions to restore the pre-determined tissue upon implantation is selected from the group consisting of inhomogeneity, anisotropy, nonlinearity, viscoelasticity, and combinations thereof.

30. The method of claim 28, wherein the tissue is articular cartilage.

31. The method of claim 28, comprising providing in the scaffold one or more cells that can develop into a pre-determined tissue.

32. The method of claim 31, wherein the one or more cells are provided in a matrix.

33. The method of claim 32, wherein the matrix comprises a gel.

34. The method of claim 32, wherein the one or more cells are selected from the group consisting of primary cells, undifferentiated progenitor cells, chondrocytes, bone-precursor cells, stem cells, cells of the periosteum, or perichondrium tissue, and combinations thereof.
